(19) United States
(12) Patent Application Publication
(10) Pub. No.: US 2015/0125548 A1
(43) Pub. Date: May 7, 2015

(54) COMBINATION NUTRITIONAL AND NUTRACEUTICAL PRODUCTS

(71) Applicant: Requis Pharmaceuticals Inc., West Chester, PA (US)

(72) Inventors: Lars Jacob Stray Knutsen, West Chester, PA (US); Judi Lois Knutsen, Cambridge (GB)

(73) Assignee: Requis Pharmaceuticals Inc., West Chester, PA (US)

(21) Appl. No.: 14/358,567

(22) PCT Filed: Nov. 13, 2012
(86) PCT No.: PCT/US12/64846

§ 371 Date: May 15, 2014

Related U.S. Application Data
(60) Provisional application No. 61/559,444, filed on Nov. 14, 2011.

(30) Foreign Application Priority Data
Nov. 13, 2012 (WO) PCTUS1264846

(51) Int. Cl. A61K 31/405; A61K 45/06; A23L 2/02; A23L 1/305; A23C 9/158; A23C 9/156; A23C 9/152; A61K 31/4045; A23L 2/52

(52) U.S. Cl. CPC A61K 31/405; A61K 31/4045; A61K 45/06; A23L 2/02; A23L 2/52; A23C 9/158; A23C 9/156; A23C 9/1526; A23L 1/3051; A23V 2002/00
USPC 424/641; 514/419; 424/729; 424/733; 424/728; 424/730

(57) ABSTRACT

The present invention is directed to nutritional and nutraceutical compositions which comprise novel mixtures of two or more indole-based dietary supplements such as L-tryptophan and melatonin, together with selected minerals and vitamins that stimulate tryptophan metabolism in the direction of serotonin and melatonin, and to their use in a preferred embodiment of the invention in a readily ingested beverage form, optionally combined with a range of other natural ingredients designed to improve the health and state of mind of human subjects. Alternative dosage forms, such as tablets for oral dosing, and dosage forms for buccal and sublingual administration, including thin films, are also incorporated in the present invention. Health improvements include the treatment of disorders of circadian rhythm, such as shift work disorder and jet lag, the induction of relaxation and a restful frame of mind, as well as providing restorative sleep function. The compositions have utility in the treatment of jet lag disorder, circadian rhythm sleep disorders including shift work disorder, and other sleep-related problems.

### CROSS REFERENCE TO RELATED APPLICATIONS

This application is the US national application of PCT/US2012/064846, filed on Nov. 13, 2012 and which claims priority to U.S. Provisional Application No. 61/559,444 filed on Nov. 14, 2011, now expired, the disclosure of which is herein incorporated by reference in its entirety.

### FIELD OF THE INVENTION

The present invention relates to nutritional and nutraceutical products, particularly to beverages, and to nutritional compositions for use in providing such products. These beverages have utility in the improvement of the health and state of mind of human subjects.

### BACKGROUND TO THE INVENTION

In contrast to the stamina, alertness, and fast pace touted by caffeinated energy drinks, a new class of beverages known as relaxation drinks promises calmness and a way to wind down when life gets stressful (see for example Stacy, S. Relaxation Drinks and Their Use in Adolescents. Journal of Child and Adolescent Psychopharmacology, 2011, 21, 605-610). A specialized subset of functional beverages aiming to produce relaxation effects is those to treat Jet Lag Disorder (JLD).

Increased prevalence of high-speed air travel, especially across international time zones, has become a feature of the modern lifestyle. Many travelers, especially business travelers, are expected to function well, despite crossing multiple time zones as part of jet travel, when they are often not working at peak performance due to JLD, and therefore can find themselves unfit for the tasks required of them because of the serious effects of circadian rhythm disturbance. Aside from sleep and stress problems, JLD can also cause fatigue, an unwell feeling, difficulty staying alert, GI issues, confusion, anxiety and amnesia.

JLD arises as a consequence of circadian misalignment that occurs after crossing time zones too rapidly for the human circadian system to keep pace. The American Academy of Sleep Medicine defines JLD as a syndrome involving insomnia or excessive daytime sleepiness following travel across at least 2 time zones. It is a recognized circadian rhythm sleep disorder characterized by insomnia or excessive daytime sleepiness (and sometimes general malaise, fatigue and somatic symptoms) associated with transmeridian jet travel.

Sleep may be prevented in human subjects if they are under stress. The latency time to falling asleep—known as sleep onset latency, is a measurable period of time. Oral dosing of L-tryptophan has been shown to alter mood in the direction of relaxation and a perception of reduced stress, owing to its incremental effects on serotonin synthesis in the central nervous system (CNS). Experimental data, including tryptophan depletion studies in human subjects, has shown that tryptophan can be an important determinant of mood, cognition, and behavior, once it is transported from plasma into brain via the Large Amino Acid Transporter (LNAA) (Richard, D. M.; Dawes, M. A.; Mathias, C. W.; Acheson, A.; Hill-Kapturczak, N.; Dougherty, D. M. International Journal of Tryptophan Research, 2009, 2, 45-60).

A rational treatment approach for JLD is now grounded in an understanding of the biology of the human circadian timekeeping system. New discoveries and awareness within the biology of circadian rhythm along with a renewed emphasis on the role of melatonin secretion in the regulation of circadian timing have provided new and accessible treatment modalities to accelerate circadian realignment, based on awareness of circadian physiology (Sack, R. L. Travel Medicine and Infectious Disease. 2009, 7, 102-10).

The science of circadian rhythm biology and a growing body of literature on human clinical research on circadian rhythm sleep disorders (CRSDs) prompted the American Academy of Sleep Medicine to convene a task force of experts to review of this important topic. The first of two publications, in addition to providing a general introduction to circadian biology, addresses “exogenous” circadian rhythm sleep disorders, including shift work disorder (SWD) and JLD (Morgenthaler, T. I. Lee-Chiong, T.; Alessi, C.; Friedman L.; Aurora, R. N.; Boehlecke, B.; Brown, T.; Chesson A. L. Jr.; Kapur, V.; Maganti, R.; Owens, J.; Pancer, J.; Swick, T. J.; Zak, R, Sleep, 2007, 30, 1445-59). The article concludes that melatonin administered at the appropriate time is indicated to reduce symptoms of JLD and improve sleep following travel across multiple time zones.

There have been at least 12 double-blind, placebo-controlled field trials of melatonin. The dose of melatonin ranged from 0.5 to 10 mg, administered at bedtime, for up to 3 days prior to departure and up to 5 days upon arrival at the destination. Studies generally demonstrated improvement in JLD symptoms with melatonin administration, although some studies were equivocal. Melatonin administered following travel improves the duration and quality of sleep, based on both subjective and objective measures of sleep. The medical literature has not produced evidence of significant risk derived from its use, but the benefits are well supported, and the safety risks from ingesting melatonin in adults appear to be low.

As early as 1997, the potential for melatonin in the treatment of CRSDs was becoming apparent. Bernd, S in Wiener Klinische Wochenschrift, 1997, 109, 714-721 reported that melatonin alone was effective in double-blind, placebo-controlled trials as a chronobiotic in circadian rhythm sleep disorders such as JLD, shift-worker sleep disorder, delayed sleep phase syndrome, and non-24-h sleep-wake disorder, and as a hypnotic for insomnia in elderly patients with a relative melatonin deficit. No mention is made however of its combination with other indole-based dietary supplements or functional foods.

New insights from the emerging field of Circadian Rhythm Science on the evaluation and treatment of CRSDs, employing the methodology of evidence-based medicine, have recently been published. These provide an overview of the general principles of circadian biology that underlie clinical evaluation and treatment, followed by a report on the accumulated evidence regarding the evaluation and treatment of SWD and JLD (Sack, R. L; Auckley, D.; Auger, R. R.; Carskadon, M. A; Wright, K. P Jr.; Vitiello, M. V; Zhdanova, I. V. Sleep, 2007, 30, 1460-83).

L-Tryptophan is an essential amino acid, meaning that it cannot be synthesized by the human body and therefore must be part of our diet. Amino acids, including tryptophan, act as building blocks in protein biosynthesis and in addition, tryptophan functions as a biochemical precursor for serotonin (5-hydroxytryptamine; 5-HT) and melatonin. After absorption, tryptophan is present in blood, around 20% as the free amino acid and the rest bound to plasma proteins. Provided there are not too many other amino acids competing for the transporters, tryptophan is transported into the CNS via the LNAA, where it is available for metabolism into 5-HT and eventually to melatonin. Tryptophan loading thereby indirectly improves sleep in adults with some sleep disturbance (Silber, B. Y.; Schmitt, J. A. J. Effects of tryptophan loading on human cognition, mood, and sleep, Neuroscience and Biobehavioral Reviews, 2010, 34, 387-407).

There is a demonstrable effect on stress symptoms through ingestion of L-tryptophan. The uptake of the serotonin precursor L-tryptophan into the brain depends on nutrients that influence the availability of tryptophan by changing the ratio of plasma tryptophan to the sum of the other large neutral amino acids (the Trp/LNAA ratio). When stress-vulnerable subjects were fed whey protein rich in -lactalbumin this key ratio increased and their cognitive performance subsequently improved. The results suggest that elevated levels of dietary tryptophan improves cognitive performance in stress vulnerable subjects via increased brain tryptophan and serotonin activities (Markus, C. R.; Berend, O.; de Haan, E. H. F. American Journal of Clinical Nutrition, 2002, 75, 1051-1056).

Tryptophan metabolism to 5-HT provides positive effects in conditions where potentially depleted 5-HT levels exist such as anxiety disorders, depression, obsessive-compulsive disorders (OCD), as well as obesity and eating disorders.

The metabolic pathway of tryptophan illustrates that L-tryptophan can metabolize to niacin, also known as vitamin B₃, initially involving the enzyme indoleamine 2,3-dihydrogenase via kynurenine. When niacin is co-dosed with tryptophan, metabolism is shunted in the direction of 5-hydroxytryptophan (5-HTP) and on to 5-HT. Niacin dosing has the effect of sparing tryptophan which can then be used to synthesize 5-HT (Beretich, G. R. Jr., Medical Hypotheses, 2005, 65, 628-629).

Pyridoxal phosphate, also known as vitamin B₆, is involved in almost all amino acid metabolism, from synthesis to breakdown. Vitamin B₆ is therefore required for the conversion of tryptophan to niacin and low vitamin B₆ status will impair this conversion. Supplementation with vitamin B₆ corrects most drug-induced disorders of tryptophan metabolism (Price, J. M. Edited by Yamada, K. Symp. Pyridoxal Enzymes, 3rd., 1968, 213).

The first step in the biosynthesis of 5-HT from tryptophan involves the enzyme tryptophan hydroxylase. When isolated from rat brain, enzymic activity was increased in the presence of mM concentrations of calcium (Hamon, M.; Bourgoin, S.; Artaud, F.; Hery, F. Journal of Neurochemistry, 1977, 28, 811-818). In addition, investigators have shown that a significant activation of this enzyme tryptophan hydroxylase was achieved by the addition of 1 mM ATP and 10 mM magnesium chloride to 100,000 g supernatant prepared from mouse midbrain. The activation produced an increase of enzyme activity by 50-70% above control (Lysz, T. W.; Sze, P. Y. J. Neuroscience Research, 1978, 3, 411-418).

Therefore calcium salts such as calcium citrate and magnesium salts such as magnesium oxide will be present in the formulation to stimulate activity of tryptophan hydroxylase, thereby increasing CNS levels of 5-HT.

In a further embodiment of this invention, a preferred indole-based dietary supplement is 5-HTP and in combination with vitamins B₃, B₆ and B₁₂, as well as selected pharmaceutically acceptable calcium and magnesium salts finds utility in the treatment of JLD, CRSDs and other sleep-related problems. JLD and CRSDs are significant sleep problems, which result in increased demand for effective sleep aids, including over-the-counter (OTC) drugs and nutraceuticals, as travelers and those working in shift patterns explore means of self-medication to induce restorative sleep function, and to improve timing of sleep as well as enhancing sleep quality, thereby improving daytime functioning. It is important to differentiate OTC and other commonly available sleep-aids and relaxation beverages from prescription-only hypnotic drugs with trade names such as Ambien™, Lunesta™ or the closely-related Zopiclone. These drugs are effective but can lead to dependency and are associated with adverse effects, such as sedation and errors in completing regular tasks the day after use, as well as potential nausea, vomiting, and confusion in the case of Ambien (zolpidem). Melatonin and L-tryptophan when used as sleep-aids would obviously tend to be much milder in effect.

A nutraceutical is a food or food product that provides health and medical benefits, including the prevention and treatment of human ailments. Most vitamins are made from “food grade” chemicals but a nutraceutical is made from pharmaceutical grade chemicals of higher purity and with more predictable and consistent results. The benefit of the nutraceutical approach in the case of treatment of JLD is that indole-based dietary supplements such as tryptophan, 5-HTP and melatonin can be combined with selected synergistic minerals and vitamins in an appealing liquid form, and presented as a beverage for ready compliance of those suffering from JLD, providing benefit, for example to transmeridian travelers.

Alternative dosage forms, such as tablets for oral dosing, and dosage forms with benefits in buccal and sublingual administration which can attenuate first-pass metabolism, including thin film formulation, are also incorporated in the present invention.

Depression is commonly associated with poor sleep (difficulty going to sleep, early waking, and general lethargy during the day). Good sleep hygiene is therefore important to help break this vicious circle, and a further embodiment of this invention is the treatment of early waking.

Furthermore the non-toxic nature of the indole-based nutraceuticals of the present invention is evidenced by the recent discovery that melatonin, L-tryptophan and vitamin B₁₂ have been shown to be important components of human breast milk, with beneficial chronobiotic effects on infants. Additionally, concentrations of these components in human breast milk show circadian oscillations and therefore vary in a diurnal fashion (Arslanoglu, S.; Bertino, E.; Nicocia, M.; Moro, G. E. Potential chronobiotic role of human milk in sleep regulation. Journal of Perinatal Medicine, 2012, 40, 1-8).

US Pat. Appl. Publ. US 2002/0004049 describes compositions comprising partially defatted meal from a plant source containing protein-bound tryptophan, preferably squash seeds, and, optionally, a carbohydrate source provided in an amount capable of facilitating transport of in vivo generated tryptophan across the blood brain barrier. Also described are dietary supplements, foods and beverages comprising the composition of the invention to improve sleep.

US Pat. Appl. Publ. US 2002/0173549 outlines a method for treating Mild Cognitive Impairment (MCI). The treatment includes administering an effective amount of a natural product that increases soluble amyloid precursor protein expression. One of the many natural products claimed is tryptophan, and a foodstuff that incorporates a natural product useful in treating MCI. US Pat. Appl. Publ. US 2010/0166916 provides nutritional compositions comprising a source of tryptophan and which nutritional compositions further comprise one or more components selected from a group consisting of: a caseinate; magnesium lactate, magnesium gluconate; zinc lactate and zinc gluconate. The application claims beverages, food products and methods of consumption.

US Pat. Appl. Publ. US 2009/0269443 discloses a process to prepare packaged pasteurized or sterilized aqueous drinkable products which comprise casein micelles (e.g. from dairy protein) and peptide fractions, which peptide fractions are rich in tryptophan, and the products so obtained. The process requires a heat preservation treatment at a specific pH.

Italian patent application, IT 2007MI1503, claiming a fatigue-reducing amino acid composition, comprises amino acids and salts useful in the reduction of muscle fatigue and dehydration in athletes. The compositions include the principal branched-chain amino acids leucine, isoleucine and valine as well as the essential amino acids lysine, threonine, histidine, phenylalanine, and tryptophan, together with sodium chloride. However, no claims for the treatment of sleep disorders are included in the application.

In KR 2009045721, sleep-improving preparations are revealed, comprising palatinose and more than one ingredient selected from bitter sesquiterpene lactones (BSL), plant extracts containing BSL, glycine and tryptophan. The compositions are claimed to have a good effect on treating sleep disorders. The invention also relates to functional foods containing the compositions.

In WO 2007063297, nutritional compositions, especially beverages comprise a source of tryptophan and one or more components selected from the group consisting of: a caseinate; magnesium lactate; magnesium gluconate; zinc lactate; and zinc gluconate. The beverages or food products may be consumed following exercise and prior to sleeping.

None of the patent applications outlined above contain claims for utility of the compositions in the treatment of CRSDs such as JLD.

### SUMMARY OF THE INVENTION

According to the present invention there is provided a nutritional and/or nutraceutical composition comprising a mixture of two or more indole-based dietary supplements such as tryptophan and/or melatonin combined with pharmaceutically-acceptable calcium and magnesium salts, as well as vitamins B₃, B₆ and B₁₂, especially in beverage form designed to improve the health and state of mind of human subjects, wherein the tryptophan can optionally be provided as the free amino acid salt thereof, and melatonin can optionally be provided in either in salt or neutral form, and the indole-derived dietary supplements may optionally be of biosynthetic or synthetic origin.

The formulations described herein are useful in the treatment of JLD and the treatment of disorders of circadian rhythm such as CRSDs, shift work disorder, as well as providing a means to access restorative sleep function, the treatment of insomnia and other sleep-related issues. Other desirable health improvements provided by the use of the formulations described herein also include the induction of a state of relaxation and a restful frame of mind and the treatment of anxiety.

Aside from the preferred admixture of L-tryptophan and melatonin, this present invention allows for the presence of two indole-based dietary supplements, including 5-hydroxytryptophan (5-HTP) in combination with a range of other substances that are perceived by consumers of functional foods to have positive effects on sleep, including, but not limited to chamomile, hops, lemon balm, passionflower, valerian, kava, rosehips, GABA, green tea extracts, including L-theanine, L-threonine, L-carnitine, phenibut (β-Phenyl-γ-aminobutyric acid), zinc, vitamin A, vitamin C, vitamin D, ginko biloba, ginseng, lemongrass, Linden, passiflora, Hawthorne Berry, Milk Thistle, Root Extract, Rhodiola Rosea extract, St. John's Wort, Mucuna Pruriens, Omega-3 fatty acids, folic acid, inositol, poppy seed extract, Resvida trans-Resveratrol, resveratrol, red wine extract (polyphenols), lavender, corydalis, cherry juice, red (rooibos) tea extracts, agave nectar and xanthan gum.

In a preferred embodiment, the composition for a single beverage dose comprises an amount of 0.01 g to 10.0 g of L-tryptophan, in combination with melatonin in the amount of 0.0001 to 0.30 g. In another embodiment, the composition will optionally contain vitamin B₃ and or vitamin B₆. Vitamin B₃ can be present in amounts ranging from about 0.002 g to 0.20 g, vitamin B₆ can be present in amounts ranging from 0.0002 g to 0.20 g and vitamin B₁₂ can be present in amounts ranging from around 0.000001 g to 0.50 g. Furthermore, vitamin B₁₂ can be dosed as cyanocobalamin (a form that the body readily converts to the active forms methylcobalamin and 5-deoxyadenosylcobalamin) and methylcobalamin, which can be be present in amounts ranging from around 0.000001 g to 0.50 g.

In another preferred embodiment, the composition will optionally contain calcium and magnesium salts. Calcium salts can be present in amounts ranging from about 0.001 g to 0.50 g, and magnesium salts can be present in amounts ranging from 0.001 g to 0.50 g.

### DETAILED DESCRIPTION OF THE INVENTION

L-Tryptophan, then, is known to be an essential amino acid that is critical in the natural production of both 5-HT and melatonin in humans—these neurotransmitters are seen as necessary for regular sleep (melatonin helps regulate the body's circadian rhythm, while 5-HT communicates a need for sleep to the brain).

Melatonin is a hormone produced by the pineal gland, a small organ in the brain which responds to light. Its production is stimulated by darkness and suppressed by light, and it is understood to be a key to helping the mind to recognize when it is time to sleep, and when to wake up.

Vitamins and minerals that have been shown to drive L-tryptophan metabolism in the direction of 5-HT are also included as a feature of this novel formulation.

Various documents including, for example, publications and patents, are recited throughout this disclosure. All such documents are hereby incorporated by reference. Trade names for products or components including various ingredients may be referenced herein. The inventors herein do not intend to be limited by materials under a certain trade name.

In the description of the invention various embodiments or individual features are disclosed. As will be apparent to the ordinarily skilled practitioner, all combinations of such embodiments and features are possible and can result in preferred executions of the present invention. The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

With respect to dosing preferences, dosage levels are developed based on typical human subjects (e.g. a 70 kg subject). If the present composition is used in other mammals or in various human subjects, it may be necessary to modify the dosage. Modification of dosages based on the needs of the subject is well within the skill of the ordinary artisan. It is therefore understood that these dosage ranges are by way of example only, and that daily administration can be adjusted depending on various factors. The specific dosage of the compounds to be administered, and the duration of treatment are interdependent. The dosage and treatment regimen will also depend upon such factors as the specific compound used, the treatment indication, the efficacy of the compound, the personal attributes of the subject (such as, for example, weight, age, gender, and medical condition of the subject), and compliance with the treatment regimen.

In a further embodiment of this invention, novel compositions of indole-based dietary supplements such as tryptophan, 5-hydroxytryptophan and melatonin in combination with niacin (also known as vitamin B₃, nicotinic acid or vitamin PP) are claimed for the treatment of sleep related problems such as CRSDs and JLD or for the induction of relaxation and a restful frame of mind in human subjects. Co-dosing of vitamins, for example niacin, assists in the metabolism of tryptophan to 5-HT in human subjects.

In a further embodiment of this invention, novel compositions of indole-based dietary supplements such as tryptophan, 5-HTP and melatonin in combination with vitamin B₆ are claimed for the treatment of insomnia and other sleep-related problems such as issues with circadian rhythm and jet lag. Co-dosing of vitamins, for example B₆, assists in the metabolism of tryptophan to 5-HT in human subjects.

These novel combinations of indole-based dietary supplements with calcium salts have utility in the treatment of insomnia and other sleep-related problems such as issues with CRSDs, shift work disorder and JLD. It is known that calcium stimulates the metabolism of tryptophan and the presence of calcium salts will likely bring about more rapid formation of 5-HT (Boadle-Biber, M. C., Biochemical Pharmacology, 1975, 24, 1455-1460).

In a further embodiment of this invention, novel compositions of indole-based dietary supplements such as tryptophan, 5-HTP and melatonin in combination with pharmaceutically acceptable calcium salts are claimed for the treatment of sleep-related problems such as CRSDs and JLD or for the induction of relaxation and a restful frame of mind in human subjects. Representative pharmaceutically acceptable calcium salts include calcium chloride, calcium tartrate, calcium maleate, calcium lactate, calcium citrate, calcium phosphate, calcium acetate, calcium carbonate, calcium hydrogen carbonate, calcium lactate calcium fumarate, calcium sulfate, calcium bromide, calcium mesylate, calcium palmoate, calcium iodide, calcium nitrate, calcium gluconate and calcium methylsulfate.

In a further embodiment of this invention, novel compositions of indole-based dietary supplements such as tryptophan, 5-HTP and melatonin in combination with pharmaceutically acceptable magnesium salts are claimed for the treatment of sleep-related problems such as CRSDs and JLD or for the induction of relaxation and a restful frame of mind in human subjects. Representative pharmaceutically acceptable magnesium salts include magnesium chloride, magnesium tartrate, magnesium maleate, magnesium lactate magnesium citrate, magnesium phosphate, magnesium phosphate monobasic, magnesium phosphate dibasic, magnesium acetate, magnesium carbonate, magnesium lactate magnesium fumarate, magnesium stearate, magnesium sulfate, magnesium bromide, magnesium mesylate, magnesium disuccinate, magnesium palmoate, magnesium iodide, magnesium nitrate and magnesium methylsulfate.

### METHODS OF USE

### EXAMPLE 1

Pyridine-3-carboxylic acid (niacin, 0.010 g), 4,5-bis(hydroxymethyl)-2- methylpyridin-3-ol (pyridoxine, 0.001 g), finely-powdered calcium carbonate (2.50 g) and magnesium oxide (0.334 g) were introduced, followed by citric acid (2.0 g) (g) followed by sweetened mango flavor powder (5.115 g). All components were certified as USP grade and were sourced from the Professional Compounding Centers of America (PCCA) of 9901 South Wilcrest Drive, Houston, Tex. 77099, USA.

The solid blend was mixed thoroughly by agitated in a large jar, and split into 10 equal amounts by weight and stored conveniently in plastic cachets. The final beverage solution was provided by combining the powder from single sachets of the blend described above with USP grade (2S)-2-amino-3-(1H-indol-3-yl)propanoic acid (L-tryptophan, 0.65 g) and N-[2-(5-methoxy-1H-indol-3-yl)ethyl]ethanamide (melatonin, 0.003 g) which had been pre-weighed into a 0.25 L sterilized plastic bottle, and adding water (240 mL). The resultant suspension was shaken well and stored in a refrigerator at 4° C. prior to controlled consumption and subsequent evaluation.

### EXAMPLE 2

USP grade (2S)-2-amino-3-(1H-indol-3-yl)propanoic acid (L-tryptophan, 3.0 g) and N-[2-(5-methoxy-1H-indol-3-yl)ethyl]ethanamide (melatonin, 0.080 g) were weighed into a sterilized glass bottle. Pyridine-3-carboxylic acid (niacin, 0.10 g) and 4,5-bis(hydroxymethyl)-2-methylpyridin-3-ol (pyridoxine, 0.010 g), calcium citrate (5.0 g) and magnesium phosphate dibasic (MgHPO₄, 2.0 g), all of USP grade, were suspended in a pasteurized milk-based chocolate flavored beverage (1.0 L, consisting of low-fat milk, sugar, cocoa processed with alkali corn starch, salt, carrageenan, vanillin, vitamin A palmitate and vitamin D₃) at room temperature. The nutraceutical beverage combination was carefully sealed and shaken utilizing a high-load orbital shaker for 1 h. The sterile liquid was dispensed in equal amounts into ten 100 mL plastic bottles, and stored in a refrigerator at 4° C. prior to evaluation as a beverage-based treatment for JLD in human subjects. Samples could readily be removed for analysis and stability testing of the beverage formula.

### EXAMPLE 3

USP grade (2S)-2-amino-3-(1H-indol-3-yl)propanoic acid (L-tryptophan, 6.0 g) and N-[2-(5-methoxy-1H-indol-3-yl)ethyl]ethanamide (melatonin, 0.160 g) were weighed into a sterilized glass bottle. Pyridine-3-carboxylic acid (niacin, 0.20 g) and 4,5-bis(hydroxymethyl)-2-methylpyridin-3-ol (pyridoxine, 0.020 g), calcium citrate (10.0 g), magnesium phosphate dibasic (MgHPO₄, 4.0 g) and vitamin B₁₂ (0.010 g) (as cyanocobalamin which converts to the active form in the body); all of USP grade, were suspended in a pasteurized mango fruit drink (2.0 L, consisting of water, mango pulp (21%), sugar, citric acid and approved flavoring) at room temperature. The nutraceutical beverage combination was carefully sealed and shaken utilizing a high-load orbital shaker for 1 h. The homogeneous sterile liquid suspension was dispensed in equal amounts into twenty 100 mL polycarbonate bottles, sealed and stored in a refrigerator prior to evaluation as a beverage-based treatment for CRSD in human subjects.

### EXAMPLE 4

USP grade (2S)-2-amino-3-(1H-indol-3-yl)propanoic acid (L-tryptophan, 3.0 g) and N-[2-(5-methoxy-1H-indol-3-yl)ethyl]ethanamide (melatonin, 0.080 g) were weighed into a sterilized glass bottle. Pyridine-3-carboxylic acid (niacin, 0.10 g) and 4,5-bis(hydroxymethyl)-2-methylpyridin-3-ol (pyridoxine, 0.010 g), calcium citrate (5.0 g) and magnesium phosphate dibasic (MgHPO₄, 2.0 g), all of USP grade, were suspended in a pasteurized orange-based beverage (1.0 L, consisting of water, orange juice and approved flavoring) at room temperature. The nutraceutical beverage combination was carefully sealed and shaken utilizing a high-load orbital shaker for 1 h. The sterile liquid was carbonated and dispensed in equal amounts into ten 100 mL polycarbonate bottles, sealed and stored in a refrigerator prior to evaluation as a beverage-based treatment for JLD in human subjects.

### EXAMPLE 5

Assessment of the effect of the specific beverage composition(s) exemplified above in objective outcomes following transmeridian air travel was carried out using questionnaires assessing time spent falling asleep (sleep onset latency), sleep quality, next-day alertness, daytime tiredness, ability to concentrate and other measures relevant to JLD.

### EXAMPLE 6

The evaluation of the effectiveness of the specific beverage composition(s) exemplified above in the treatment of JLD in human subjects is carried out utilizing the protocol as described in Claustrat, B, Brun, J, David, M, Sassolas, G, and Chazot, G. Biol. Psychiatry, 1992, 32, 705-711.

### EXAMPLE 7

The evaluation of the effectiveness of the specific beverage composition(s) in the treatment of cognitive effects in stressed human subjects is carried out utilizing the protocol as described in Markus, C. R.; Berend, O.; de Haan, E. H. F. Am. J. Clin. Nutrition, 2002, 75, 1051-1056.

1. A nutritional composition for circadian rhythm sleep disorders comprising at least two indole-based dietary supplements as active ingredients.

2. Compositions according to claim 1 which contain a combination of at least two indole-based dietary supplements wherein each indole-based dietary supplement is selected from the group consisting of melatonin, 5-hydroxytryptophan and tryptophan.

3. Compositions according to claim 1 which contain both L-tryptophan and melatonin as the indole-based dietary supplements.

4. Compositions according to claim 3 further containing pharmaceutically acceptable salts selected from the group consisting of calcium salts, magnesium salts, niacin, vitamin B₆, vitamin B₁₂, and combinations thereof to enhance the effect of the indole-based dietary supplements.

5. Compositions according to claim 3 further contain compounds selected from the group consisting of vitamins B₃, B₆, B₁₂, niacin, and combinations thereof.

6. Compositions according to claim 3 further containing substances which have a positive effect on sleep selected from the group consisting of chamomile, hops, lemon balm, passionflower, valerian, kava, rosehips, GABA, green tea extracts, including L-theanine, L-threonine, L-carnitine, phenibut (β-Phenyl-γ-aminobutyric acid), zinc, vitamin A, vitamin C, vitamin D, ginko biloba, ginseng, lemongrass, Linden, passiflora, Hawthorne Berry, Milk Thistle, Root Extract, Rhodiola Rosea extract, St. John's Wort, Mucuna Pruriens, Omega-3 fatty acids, folic acid, inositol, poppy seed extract, Resvida trans-Resveratrol, resveratrol, red wine extract (polyphenols), lavender, corydalis, cherry juice, red (rooibos) tea extracts, agave nectar, and combinations thereof.

7. A beverage product for the treatment of sleep disorders comprising at least two indole-based dietary supplements wherein said treatment improves symptoms associated with circadian rhythm sleep disorders, shift work disorders, or jet lag disorders to restore sleep function in humans.

8. A nutritional composition for the induction of restorative sleep comprising at least two indole-based dietary supplements in liquid or solid formulation wherein said composition reduces the symptoms of shift work disorder or jet lag disorder.

9. Compositions according to claim 8 which contain a combination of at least two indole-based dietary supplements wherein each indole-based dietary supplement is selected from the group consisting of melatonin, 5-hydroxytryptophan and tryptophan, except when in the presence of L-theanine, GABA or Phenibut.

10. A method for improving circadian rhythm sleep disorders in a human subject comprising administering a composition in claim 3 in an amount sufficient to treat symptoms associated with circadian rhythm sleep disorders.

11. The method of claim 10 wherein the circadian rhythm sleep disorder is jet lag disorder.

12. The method of claim 10 wherein the circadian rhythm sleep disorder is shift work disorder.

13. The method of claim 10 wherein an administration route is selected from the group consisting of oral buccal, sublingual, and combinations thereof.

14. The method of claim 10 wherein said composition is in beverage form, tablet form, specialized dosage forms or thin film.

15. A method for treating early waking comprising administering a composition in claim 3.

16. A method for inducing restorative sleep function in a human subject comprising administering a composition in claim 3 in a human subject.

17. A method for improving circadian rhythm sleep disorders in non-human subjects comprising administering a composition in claim 3 in a non-human subject.
